(19) United States
(12) Patent Application Publication
(10) Pub. No.: US 2010/0136072 A1
(43) Pub. Date: Jun. 3, 2010

(54) Polymeric Coatings that Inactivate Viruses and Bacteria

(75) Inventors: Jayanta Haldar, Belmont, MA (US); Deqiang An, Shanghai (CN); Luis Álvarez de Cienguegos, Granada (ES); Jianzhu Chen, Brookline, MA (US); Alexander M. Klibanov, Boston, MA (US)

Correspondence Address: Pabst Patent Group LLP, 1545 PEACHTREE STREET NE, SUITE 320, ATLANTA, GA, 30309, US

(73) Assignee: Massachusetts Institute of Technology, Massachusetts (US)

(21) Appl. No.: 12/514,101

(22) PCT Filed: Nov. 8, 2007
(86) PCT No.: PCT/US07/84149

§ 371 Date: May 8, 2009

Related U.S. Application Data
(60) Provisional application No. 60/864,967, filed on Nov. 8, 2006.

(51) Int. Cl. A61K 31/785; A01N 25/34; A01P 1/00; A61P 31/12; A01N 43/00

(52) U.S. Cl. 424/402; 424/78.35; 424/411

(57) ABSTRACT

Hydrophobic polymeric coatings which can be non-covalently applied to solid surfaces such as metals, plastics, glass, polymers, textiles, and other substrates such as fabrics, gauze, bandages, tissues, and other fibers, in the same manner as paint, for example, by brushing, spraying, or dipping, to make the surfaces virucidal and bactericidal, have been developed.

This application claims priority to U.S. Ser. No. 60/864,967 filed Nov. 8, 2006.

### GOVERNMENT SUPPORT

This invention was made with government support under Contract DAAD-19-02-D-0002 awarded by the U.S. Army through the Institute for Soldier Nanotechnologies at MIT. The government has certain rights in the invention.

### FIELD OF THE INVENTION

This application relates to polymeric coatings (also referred to as “paints”) that inactivate viruses and bacteria, and methods of use thereof.

### BACKGROUND OF THE INVENTION

There is a keen interest in materials capable of killing harmful microbes, especially materials that could be used to coat surfaces of common objects touched by people in everyday lives, e.g., door knobs, children toys, computer keyboards, telephones, etc., to render them antiseptic and thus unable to transmit viral and bacterial infections. Since ordinary materials are not antimicrobial, their modification is required. For example, surfaces chemically modified with polyethylene glycol) and certain other synthetic polymers can repel, although not kill, microorganisms (Bridgett, M. J., et al, S. P. (1992) Biomaterials 13, 411-416. Arciola, C. R., et al Alvergna, P., Cenni, E. & Pizzoferrato, A. (1993) Biomaterials 14, 1161-1164. Park, K. D., Kim, Y. S., Han, D. K., Kim, Y. H., Lee, E. H. B., Suh, H. & Choi, K. S. (1998) Biomaterials 19, 851-859.) See also U.S. Pat. No. 5,783,502 to Swanson which describes reagents and methods for modifying a fabric substrate in order to inactivate virus, particularly lipid-enveloped viruses, where the substrates are modified by photochemically immobilizing hydrophilic polymers containing both quaternary ammonium groups and hydrocarbon chains, resulting in a localized surfactancy capable of disrupting lipid-enveloped viruses upon contact with the substrate. WO 1999/40791 by Surfacine Development Co., which describes a composition that, when applied to a substrate, forms an adherent, transparent, water insoluble polymeric film on the substrate surface that provides sustained antimicrobial and antiviral action for prolonged periods, without the necessity for reapplication. The coating allegedly provides surface disinfecting action by a contact killing mechanism, and does not release its components into contacting solution at levels that would result in solution disinfection. The composition comprises a combination of an organic biguanide polymer and an antimicrobial metallic material. The polymer must be capable of reversibly binding or complexing the metallic material and insinuating the metallic material into the cell membrane of the microorganism in contact with it.

Alternatively, materials can be impregnated with antimicrobial agents, such as antibiotics, quarternary ammonium compounds, silver ions, or iodine, that are gradually released into the surrounding solution over time and kill microorganisms there (Medlin, J. (1997) Environ. Health Persp. 105, 290-292; Nohr, R. S. & Macdonald, G. J. (1994) J. Biomater. Sci., Polymer Edn. 5, 607-619 Shearer, A. E. H., et al (2000) Biotechnol. Bioeng. 67, 141-146). U.S. Pat. No. 5,437,656 to Shikani et al. describes an anti-infective coating on the metal which is complexed with an iodine solution. See also U.S. Pat. No. 6,939,569 to Green et al. and U.S. Patent Application Publication No. 2003/0091641 by Tiller, et al., which describes bactericidal compositions comprising a polymeric compound such as a hydrophobic polycation which can be covalently bonded to a substrate material or sprayed, immersed, dipped, painted, bonded or adhered to a substrate.

Although these strategies have been verified in aqueous solutions containing bacteria, they would not be expected to be effective against airborne bacteria in the absence of a liquid medium. This is especially true for release-based materials, which may also be liable to become impotent when the leaching antibacterial agent is exhausted.

Infection is a frequent complication of many invasive surgical, therapeutic and diagnostic procedures. For procedures involving implantable medical devices, avoiding infection can be particularly problematic because bacteria can develop into biofilms, which protect the microbes from clearing by the subject's immune system and from the action of drugs. As these infections are difficult to treat with antibiotics, removal of the device is often necessitated, which can be traumatic to the patient and increase the medical cost.

Since the difficulties associated with eliminating biofilm-based infections are well-recognized, a number of technologies have developed to treat surfaces or fluids bathing surfaces to prevent or impair biofilm formation. For example, various methods have been employed to coat the surfaces of medical devices with antibiotics (See e.g. U.S. Pat. Nos. 4,107,121; 4,442,133; 4,895,566; 4,917,686; 5,013,306; 4,952,419; 5,853,745; and 5,902,283) and other bacteriostatic compounds (See e.g. U.S. Pat. Nos. 4,605,564; 4,886,505; 5,019,096; 5,295,979; 5,328,954; 5,681,575; 5,753,251; 5,770,255; and 5,877,243).

Infectious organisms are ubiquitous in the medical environment, despite vigorous efforts to maintain antisepsis. The presence of these organisms can result in infection of hospitalized patients and medical personnel. These infections, termed nosocomial, often involve organisms more virulent and more unusual than those encountered outside the hospital. In addition, hospital-acquired infections are more likely to involve organisms that have developed resistance to a number of antibiotics. Although cleansing and anti-bacterial regimens are routinely employed, infectious organisms readily colonize a variety of surfaces in the medical environment, especially those surfaces exposed to moisture or immersed in fluid. Even barrier materials, such as gloves, aprons and shields, can spread infection to the wearer or to others in the medical environment. Despite sterilization and cleansing, a variety of metallic and non-metallic materials in the medical environment can retain dangerous organisms trapped in a biofilm, thence to be passed on to other hosts.

Any agent used to impair biofilm formation in the medical environment must be safe to the user. Certain biocidal agents, in quantities sufficient to interfere with biofilms, also can damage host tissues. Antibiotics introduced into local tissue areas can induce the formation of resistant organisms which can then form biofilm communities whose planktonic microorganisms would likewise be resistant to the particular antibiotics. Any anti-biofilm or antifouling agent must furthermore not interfere with the salubrious characteristics of a medical device. Certain materials are selected to have a particular type of operator manipulability, softness, water-tightness, tensile strength or compressive durability, characteristics that cannot be altered by an agent added for anti-microbial effects.

As a further problem, it is possible that materials added to the surfaces of implantable devices to inhibit contamination and biofilm formation may be thrombogenic. Some implantable materials are themselves thrombogenic. For example, it has been shown that contact with metal, glass, plastic or other similar surfaces can induce blood to clot. Heparin compounds, which are known to have anticoagulant effects, have therefore been applied to certain medical devices prior to implantation. However, there are few known products in the medical arsenal whose antimicrobial effects are combined with antithrombogenic effects. This combination would be particularly valuable to treat those medical devices that reside in the bloodstream, such as heart valves, artificial pumping devices (“artificial hearts” or left ventricular assist devices), vascular grafting prostheses and vascular stents. In these settings, clot formation can obstruct the flow of blood through the conduit and can furthermore break off pieces called emboli that are carried downstream, potentially blocking circulation to distant tissues or organs.

Viruses are an even bigger problem than bacteria since there are so few antiviral products and no general antiviral products. Viral epidemics can spread rapidly, and through air, water, or via direct contamination. For example, influenza virus causes one of the most prevalent human infections: in a typical year, about 15% of the U.S. population is infected, resulting in up to 40,000 deaths and 200,000 hospitalizations (http://www.cdc.gov/flu). Furthermore, an influenza pandemic (when a new strain of the virus, to which humans have no immunity, acquires the ability to readily infect people), assuming the estimated mortality rate of the 1918 Spanish flu pandemic (Wood et al. (2004) Nature Rev Microbiol 2:842-847), might kill some 75 million people worldwide.

Influenza (as many other diseases) typically spreads when aerosol particles containing the virus, exhaled or otherwise emitted by an infected person, settle onto surfaces subsequently touched by others (Wright et al. (2001) in Fields Virology, 4^{th} edition, eds. Knipe D M, Howley P M (Lippincott, Philadelphia, Pa.), pp 1533-1579). Hence this spread of infection, in principle, could be prevented if common things encountered by people are coated with “paints” that inactivate influenza virus.

There exists, therefore, a need to be able to render general surfaces bactericidal and/or virucidal.

It is therefore an object of the present invention to provide materials and methods of use thereof to provide bactericidal and/or virucidal surfaces.

### SUMMARY OF THE INVENTION

Hydrophobic polymeric coatings which can be non-covalently applied to solid surfaces such as metals, plastics, glass, polymers, and other substrates such as fabrics, gauze, bandages, tissues, and other fibers, in the same manner as paint, for example, by brushing, spraying, or dipping, to make the surfaces virucidal and bactericidal, have been developed.

Polymers are preferably hydrophobic, water-insoluble, charged, and can be linear or branched. Preferred polymers include linear or branched derivatives of polyethyleneimine. Higher molecular weight polymers are more virucidal. Preferred polymers have weight average molecular weights of greater than 20 kDa, preferably greater than 50 kDa, more preferably greater than 100 kDa, more preferably greater than 200 kDa, and most preferably greater than 750 kDa. As demonstrated by the examples, suitable polymers include a 217 kDa polyethylenimine (PEI), prepared from commercially available 500 kDa poly(2-ethyl-2-oxazoline) by acid hydrolysis and then quaternized by dodecylation, followed by methylation, as described in Klibanov et al., Biotechnology Progress, 22(2), 584-589, 2006). The structure of this polymer is shown below:

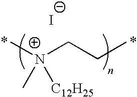

Other hydrophobic polycationic coatings which can be used include the polymers shown below:

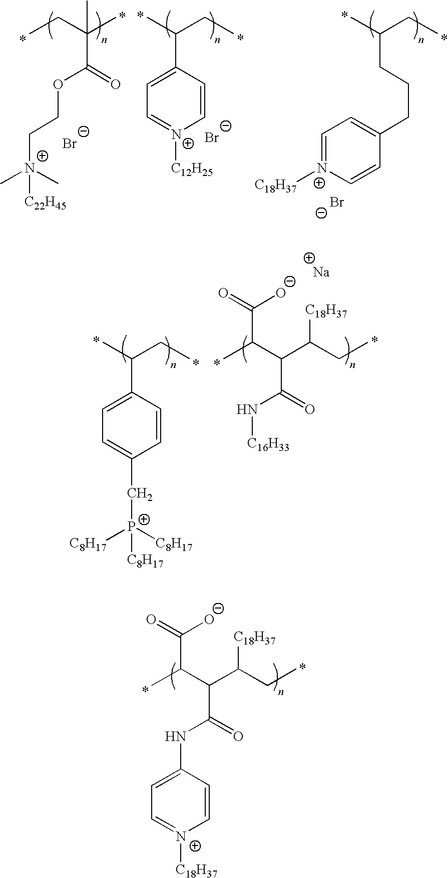

The coating polymer can be dissolved in a solvent, preferably an organic solvent such as butanol, and applied to a substrate, for example, by brushing or spraying the solution and then drying to remove the solvent.

As demonstrated by the examples, painting a glass slide with branched or linear N,N-dodecyl,methyl-PEIs and other hydrophobic PEI derivatives results in killing of influenza virus with essentially a 100% efficiency (at least a 2-log, more preferably 3-log, most preferably at least a 4-log reduction in the viral titer) within minutes, as well as the airborne human pathogenic bacteria Escherichia coli and Staphylococcus aureus. For most of the coating polyions this virucidal action is shown to occur on contact, i.e., solely by the polymeric chains anchored to the slide surface; although for others, the polyion leaching from the painted surface may contribute to virucidal activity. A relationship between the structure of the derivatized PEI and the resultant virucidal activity of the painted surface has been elucidated. The polymer should be sufficiently hydrophobic to be insoluble in water and thus remain coated on the surface of the substrate. The positive charge appears to be desirable, but is not required as shown by the negatively charged and zwitterionic hydrophobic polymers. The coated slides were shown to be virucidal to influenza A/WSN/33(H1N1) and influenza A/Victoria/3175 (H3N2) strains; A/Wuhan/359/95 (H3N2)-like wild type influenza virus and an oseltamivir-resistant variant, Glu119Val; and A/turkey/Minnessota/833/80 (H4N2) wild type influenza virus and three neuraminidase inhibitor-resistant variants, Glu119Asp, Glu119Gly, and Arg292Lys.

### BRIEF DESCRIPTION OF THE DRAWINGS

FIG. 1A is a schematic representation of the N-dodecylation and subsequent N-methylation of branched PEI. In the case of the resulting product, labeled “1a-c”, the letters a, b, and c are used to indicate that the N,N-dodecyl,methyl-polycations were prepared from 750-kDa, 25-kDa, and 2-kDa PEIs, respectively. FIG. 1B contains five (5) chemical structures of linear PEI-based polymers synthesized, as described in the examples. In the case of the polymer labeled “2a-c”, the letters a, b, and c indicate that the N,N-dodecyl,methyl-polycations were prepared from 217-kDa, 21.7-kDa, and 2.17-kDa PEIs, respectively. For the polymers labeled “3”, “4”, “5” or “6”, only a 217-kDa PEI was employed.

FIG. 2 is a graph of the time course (minutes) of inactivation of influenza virus (WSN strain) by a glass slide painted with Structure 2a at room temperature.

FIG. 3 is a graph of the virucidal activity against influenza virus (WSN strain) of glass slides painted with Structure 2a, 4, or 5 after different time periods (5, 30 or 120 minutes) of exposure at room temperature.

### DETAILED DESCRIPTION

### I. Virucidal Polymeric Coatings

A. Polymers

### DEFINITIONS

An amphipathic molecule or compound is an art recognized term where one portion of the molecule or compound is hydrophilic and another portion is hydrophobic. An amphipathic molecule or compound has a portion which is soluble in aqueous solvents, and a portion which is insoluble in aqueous solvents.

The terms “hydrophilic” and “hydrophobic” are art-recognized and mean water-loving and water-hating, respectively. In general, a hydrophilic substance will dissolve in water, and a hydrophobic one will not.

The term “water insoluble” as generally used herein means that the polymer has a solubility of less than approximately 0.1% (w/w) in water under standard conditions at room temperature or body temperature.

The term “ligand” refers to a compound that binds at the receptor site.

The term “heteroatom” as used herein means an atom of any element other than carbon or hydrogen. Preferred heteroatoms are nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (insert sigma) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), where σ[P] indicates para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C.₃₀ for branched chain), and more preferably 20 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as “alkyl” is a lower alkyl.

The term “aralkyl”, as used herein, refers to an alkyl group substituted with an aryl group (e.g., an aromatic or hetero aromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines.

The term “acylamino” is art-recognized and refers to a moiety that can be represented by the general formula:

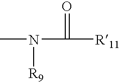

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

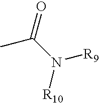

wherein R₉, R₁₀ are as defined above.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are as defined above. Representative alkylthio groups include methylthio, ethyl thio, and the like.

The term “carbonyl” is art recognized and includes such moieties as can be represented by the general formula:

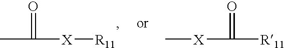

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is oxygen, and R₁₁ is as defined above, the moiety is referred to herein as a carboxyl group, and particularly when R₁₁ is a hydrogen, the formula represents a “carboxylic acid”. Where X is oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thioester.” Where X is a sulfur and R₁₁ is hydrogen, the formula represents a “thiolcarboxylic acid.” Where X is a sulfur and R_{11′} is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula:

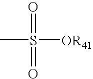

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain the groups, respectively.

The term “sulfate” is art recognized and includes a moiety that can be represented by the general formula:

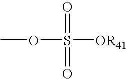

in which R₄₁ is as defined above.

The term “sulfonylamino” is art recognized and includes a moiety that can be represented

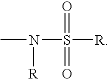

The term “sulfamoyl” is art-recognized and includes a moiety that can be represented by

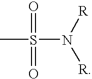

The term “sulfonyl”, as used herein, refers to a moiety that can be represented by the general formula:

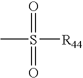

in which R₄₄ is hydrogen, alkyl, alkenyl, alkynyl, cycloalkyl, heterocyclyl, aryl, or heteroaryl.

The term “sulfoxido” as used herein, refers to a moiety that can be represented by the general formula:

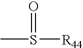

in which R₄₄ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, aminoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This polymers described herein are not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2nd ed.; Wiley: New York, 1991).

### Hydrophobic, Water Insoluble Polymers

The polymers used to form the coatings described herein are preferably hydrophobic, water-insoluble, charged, and can be linear or branched. Preferred polymers include linear or branched derivatives of polyethyleneimine. The polymer may be positively charged, negatively charged, or zwitterionic.

The molecular weight of the deposited polymer was found to be important for the antiviral and antibacterial properties of the surface. Higher molecular weight polymers are generally more virucidal. Preferred polymers have weight average molecular weights of greater than 20 kDa, preferably greater than 50 kDa, more preferably greater than 100 kDa, more preferably greater than 200 kDa, and most preferably greater than 750 kDa.

As demonstrated by the examples, suitable polymers include a 217 kDa polyethylene imine (PEI), prepared from commercially available 500 kDa poly(2-ethyl-2-oxazoline) by acid hydrolysis and then quaternized by dodecylation, followed by methylation as described in Klibanov et al., Biotechnology Progress, 22(2), 584-589, 2006). The structure of this polymer is shown below:

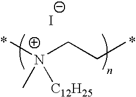

Other hydrophobic polycationic coatings which can be used include the polymers shown below:

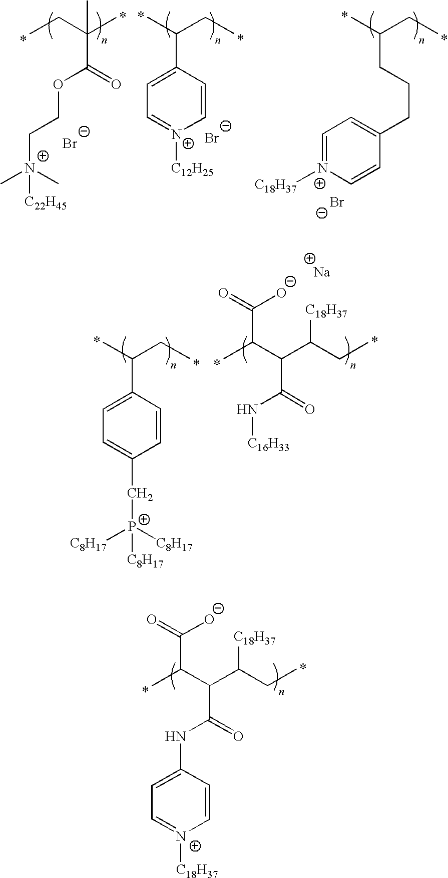

Contemplated equivalents of the polymers described above include polymers which otherwise correspond thereto, and which have the same general properties thereof, wherein one or more simple variations of substituents are made which do not significantly adversely affect the bactericidal or virucidal efficacy of the resulting polymeric coating. In general, the compounds may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

The polymer has a molecular weight of at least 10,000 g/mol, more preferably 100,000 g/mol, and most preferably 150,000 g/mol.

In certain embodiments, the compound applied to the surface is represented by the formula I:

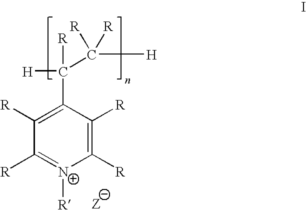

wherein R represents individually for each occurrence hydrogen, alkyl, alkenyl, alkynyl, acyl, aryl, carboxylate, alkoxycarbonyl, aryloxycarbonyl, carboxamido, alkylamino, acylamino, alkoxyl, acyloxy, hydroxyalkyl, alkoxyalkyl, aminoalkyl, (alkylamino)alkyl, thio, alkylthio, thioalkyl, (alkylthio)alkyl, carbamoyl, urea, thiourea, sulfonyl, sulfonate, sulfonamido, sulfonylamino, or sulfonyloxy;

R′ represents independently for each occurrence alkyl, an alkylidene tether to a surface, or an acyl tether to a surface;

Z represents independently for each occurrence Cl, Br, or I; and

n is an integer less than or equal to about 1500.

B. Solvents

The polymers are preferably hydrophobic and water-insoluble, and therefore are dissolved in an organic solvent, such as butanol, ethanol, methanol, butane, or methyl chloride, for application. The polymer solution should contain an effective amount of polymer to produce a virucidal, and optionally bactericidal, coating on a surface to be coated.

C. Substrates and Devices to be Coated

A “coating” refers to any temporary, semipermanent or permanent layer, covering or surface, akin to paints. The coating should be of sufficient thickness to make the surface to which the coating is applied virucidal and optionally bactericidal.

The polymer solutions can be applied to a variety of substrates to form a coating. Suitable substrates, include, for example, metal, ceramic, polymeric, and fiber, both natural and synthetic. The surfaces of the items can be coated with a polymeric coating, formed from a polymer solution containing an effective amount of a hydrophobic, water insoluble polymer polymer to form a coating having virucidal and optionally bactericidal properties.

The coatings can be applied to the surface of any material or item which needs to be virucidal and, optionally, bactericidal. Typically, items that need to be virucidal and, optionally, bactericidal include items that are handled by or that come into contact with individuals.

The items to be coated include, but are not limited to, household items, including children's toys, bathroom fixtures, counter and table tops, handles, computers, clothing, paper products, windows, doors and interior walls.

In another embodiment, the surface to be coated is the surface of an item of military gear.

Coatings may also be utilized in agricultural settings, including animal feeding and watering devices, and processing facilities. For example, in one embodiment coating of equipment used in the feeding or processing of chickens may be useful to inhibit the transmission of avian flu.

Other suitable surfaces to be coated include surfaces of items used in medical settings, including, but limited to, tissues, implants, bandages or wound dressings, medical drapes, or medical devices.

“Dressing” refers to any bandage or covering applied to a lesion or otherwise used to prevent or treat infection. Examples include wound dressings for chronic wounds (such as pressure sores, venous stasis ulcers and burns) or acute wounds and dressings over percutaneous devices such as IVs or subclavian lines intended to decrease the risk of line sepsis due to microbial invasion. For example, the compositions could be applied at the percutaneous puncture site, or could be incorporated in the adherent dressing material applied directly over the entry site.

An “implant” is any object intended for placement in a human body that is not a living tissue. Implants include naturally derived objects that have been processed so that their living tissues have been devitalized. As an example, bone grafts can be processed so that their living cells are removed, but so that their shape is retained to serve as a template for ingrowth of bone from a host. As another example, naturally occurring coral can be processed to yield hydroxyapatite preparations that can be applied to the body for certain orthopedic and dental therapies. An implant can also be an article comprising artificial components. The term “implant” can be applied to the entire spectrum of medical devices intended for placement in a human body.

“Medical device” refers to a non-naturally occurring object that is inserted or implanted in a subject or applied to a surface of a subject. Medical devices can be made of a variety of biocompatible materials, including: metals, ceramics, polymers, gels and fluids not normally found within the human body. Medical devices include scalpels, needles, scissors and other devices used in invasive surgical, therapeutic or diagnostic procedures; implantable medical devices, including artificial blood vessels, catheters and other devices for the removal or delivery of fluids to patients, artificial hearts, artificial kidneys, orthopedic pins, plates and implants; catheters and other tubes (including urological and biliary tubes, endotracheal tubes, peripherably insertable central venous catheters, dialysis catheters, long term tunneled central venous catheters peripheral venous catheters, short term central venous catheters, arterial catheters, pulmonary catheters, Swan-Ganz catheters, urinary catheters, peritoneal catheters), urinary devices (including long term urinary devices, tissue bonding urinary devices, artificial urinary sphincters, urinary dilators), shunts (including ventricular or arterio-venous shunts); prostheses (including breast implants, penile prostheses, vascular grafting prostheses, heart valves, artificial joints, artificial larynxes, otological implants), vascular catheter ports, wound drain tubes, hydrocephalus shunts, pacemakers and implantable defibrillators, and the like. Other examples will be readily apparent to practitioners in these arts.

Surfaces found in the medical environment include also the inner and outer aspects of pieces of medical equipment, medical gear worn or carried by personnel in the health care setting. Such surfaces can include counter tops and fixtures in areas used for medical procedures or for preparing medical apparatus, tubes and canisters used in respiratory treatments, including the administration of oxygen, of solubilized drugs in nebulizers and of anesthetic agents. Also included are those surfaces intended as biological barriers to infectious organisms in medical settings, such as gloves, aprons and faceshields. Other such surfaces can include handles and cables for medical or dental equipment not intended to be sterile. Additionally, such surfaces can include those non-sterile external surfaces of tubes and other apparatus found in areas where blood or body fluids or other hazardous biomaterials are commonly encountered.

Surfaces in contact with liquids may be coated and include reservoirs and tubes used for delivering humidified oxygen to patients and dental unit waterlines.

Other surfaces related to health include the inner and outer aspects of those articles involved in water purification, water storage and water delivery, and those articles involved in food processing. Surfaces related to health can also include the inner and outer aspects of those household articles involved in providing for nutrition, sanitation or disease prevention. Examples can include food processing equipment for home use, materials for infant care, tampons and toilet bowls.

The polymer coating can also be incorporated into glues, cements or adhesives, or in other materials used to fix structures within the body or to adhere implants to a body structure. Examples include polymethylmethacrylate and its related compounds, used for the affixation of orthopedic and dental prostheses within the body.

In one embodiment, compounds can be applied to or incorporated in certain medical devices that are intended to be left in position permanently to replace or restore vital functions such as ventriculoatrial, ventriculoperitoneal and dialysis shunts, and heart valves.

Other medical devices which can be coated include pacemakers and artificial implantable defibrillators, infusion pumps, vascular grafting prostheses, stents, suture materials, and surgical meshes.

Implantable devices intended to restore structural stability to body parts can be coated. Examples include implantable devices used to replace bones or joints or teeth.

Certain implantable devices are intended to restore or enhance body contours for cosmetic or reconstructive applications. Examples include breast implants, implants used for craniofacial surgical reconstruction and tissue expanders.

Insertable devices include those objects made from synthetic materials applied to the body or partially inserted into the body through a natural or an artificial site of entry. Examples of articles applied to the body include contact lenses, stoma appliances, artificial larynx, endotracheal and tracheal tubes, gastrostomy tubes, biliary drainage tubes and catheters. Some examples of catheters that may be coated include peritoneal dialysis catheters, urological catheters, nephrostomy tubes and suprapubic tubes. Other catheter-like devices exist that may be coated include surgical drains, chest tubes and hemovacs.

Dressing materials and glues or adhesives used to stick the dressing to the skin may be coated.

These above examples are offered to illustrate the multiplicity of applications of compounds. Other examples will be readily envisioned by skilled artisans in these fields. The examples given above represent embodiments where the technologies are understood to be applicable. Other embodiments will be apparent to practitioners of these and related arts. Embodiments can be compatible for combination with currently employed antiseptic regimens to enhance their antimicrobial efficacy or cost-effective use. Selection of an appropriate vehicle for bearing a compound will be determined by the characteristics of the particular use.

### II. Methods of Application and Use

The polymer coatings are typically applied to the surface to be coated by dissolving a polymer in an appropriate, preferably organic solvent, and applying by spraying, brushing, dipping, painting, or other similar technique. The coatings are deposited on the surface and associate with the surfaces via non-covalent interactions.

In some embodiments, the surface may be pretreated with an appropriate solution or suspension to modify the properties of the surface, and thereby strengthen the non-covalent interactions between the modified surface and the coating.

The polymer solution is applied to a surface at an appropriate temperature and for a sufficient period of time to form a coating on the surface, wherein the coating is effective in forming a virucidal and optionally a bactericidal surface. Typical temperatures include room temperature, although higher temperatures may be used. Typical time periods include 5 minutes or less, 30 minutes or less, 60 minutes or less, and 120 minutes or less. In some embodiments the solution can be applied for 120 minutes or longer to form a coating with the desired virucidal activity. However, preferably shorter time periods are used.

The coatings are applied in an effective amount to form a virucidal coating. As used herein, the term “virucidal” means that the polymer coating produces a substantial reduction in the amount of active virus present on the surface, preferably at least one log kill, preferably at least two long kill, when an aqueous viral suspension or an aerosol is applied at room temperature for a period of time, as demonstrated by the examples. In more preferred applications, there is at least a three log kill, most preferably a four-log kill. Although 100% killing is typically desirable, it is generally not essential. Preferably the virus to be inactivated is an enveloped virus. In one embodiment, the coating is applied to inactivate the influenza virus.

Influenza A virus is a ubiquitous and insidious human pathogen infecting tens of millions of people yearly. Particularly troublesome is the threat of another influenza pandemic which occurs when a new, likely avian strain of influenza virus, to which humans have no immunity, becomes infective to people.

Influenza viruses are mainly spread from person to person through droplets produced while coughing or sneezing. However, the viruses can also be transmitted when a person touches respiratory droplets settled on an object before transfer to mucosal surfaces. This mode of transmitting the infection should be interrupted if the object can inactivate influenza viruses.

The compositions and methods of manufacture and use thereof will be further understood by reference to the following non-limiting examples.

### EXAMPLES

### Example 1

### Preparation and Testing of Polymeric Coatings

Materials and Methods

Commercial Chemicals. Branched polyethylenimine (PEI, M_{w} values of 750, 25, and 2 kDa), poly(2-ethyl-2-oxazoline) (M_{w} values of 500, 50, and 5 kDa), organic solvents, and all low-molecular-weight chemicals were purchased from Sigma Aldrich Chemical Co. and used without further purification.

Bacteria and Media. The bacterial strains employed were Staphylococcus aureus (ATCC 33807) and Escherichia coli (E. coli genetic stock center, CGSC4401). Yeast-dextrose broth contained (per liter of deionized water): 10 g of peptone, 8 g of beef extract, 5 g of NaCl, 5 g of glucose, and 3 g of yeast extract (Lüscher-Mattli M (2000) Arch Virol 145:2233-2248). Phosphate-buffered saline (PBS) contained 8.2 g of NaCl and 1.2 g of NaH₂PO₄.H₂O per liter of deionized water. The pH of the PBS solution was adjusted to 7.0 with 1 N aqueous NaOH. Both solutions were autoclaved for 20 min prior to use.

Cells and Viruses. MDCK cells were obtained from the ATCC. They were grown at 37° C. in a humidified-air atmosphere (5% CO₂/95% air) in Dulbecco's modified Eagle's (DME-Hepes) medium supplemented with 10% heat-in-activated fetal calf serum (GIRGO 614), 100 U/ml penicillin G, 100 μg/ml streptomycin, and 2 mM L-glutamine.

Plaque-purified influenza A/WSN/33 (H1N1) strain was grown in a confluent monolayer of MDCK cells by infecting them with WSN at a multiplicity of infection (MOI) of 0.001 at room temperature for 1 h. The virus was then incubated with a growth medium (E4GH) containing 0.3% BSA at 37° C. in a humidified-air atmosphere (5% CO₂/95% air) for 2 days. The supernatants were harvested from infected cultures, and the virus was stored at −80° C. Its titer was assayed by a plaque-forming assay in MDCK cells (Cunliffe et al. (1999) Appl Environ Microbiol 65:4995-5002). Influenza A/Victoria/3/75 (H3N2) strain was obtained from Charles River Laboratories. A/Wuhan/359/95 (H3N2)-like wild type influenza virus and its oseltamivir-resistant variant carrying the Glu 119Val mutation in the neuraminidase; A/turkey/Minnesota/833/80 (H4N2) wild type; and three neuraminidase inhibitor-resistant variants (Glu119Asp, Glu119Gly, and Arg292Lys) were obtained from the U.S. Center for Disease Control and Prevention (“CDC”).

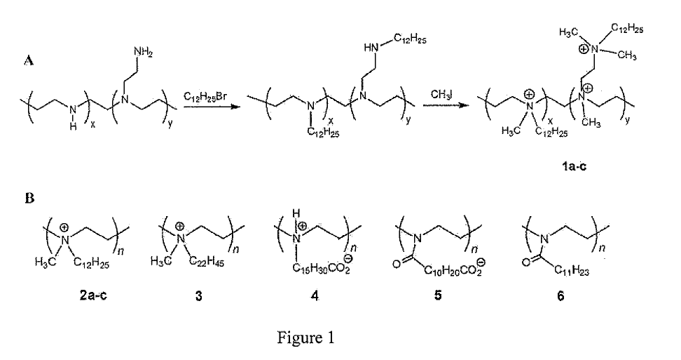

Syntheses. Branched N,N,-dodecyl,methyl-PEIs (1a, 1b, and 1c) (FIG. 1A) (prepared from branched PEIs of M_{w} of 750, 25, and 2 kDa, respectively) were synthesized (FIG. 1) and characterized as described by Park et al. (2006) Biotechnol Progr 22:584-589.

Long linear N,N-dodecyl,methyl-PEI (2a) (FIG. 1A) (from 217-kDa linear PEI) was prepared by first fully deacylating commercial poly(2-ethyl-2-oxazoline) as previously described (Ge et al. (2003) Proc Natl Acad Sci USA 100:2718-2723). The resultant protonated PEI was dissolved in water and neutralized with excess of aqueous KOH to precipitate the polymer. The latter was isolated by filtration, washed with deionized water until the pH became neutral, and dried under vacuum. Yield: 1.25 g (97%). ¹H NMR (CDCl₃): δ=2.72 (s, 4H, NCH₂CH₂N), 1.71 (s, 1H, NH) (NMR spectra here and henceforth were recorded using a Varian Mercury 300-MHz NMR spectrometer). Next, 2.0 g (47 mmol of the monomeric units) of the PEI prepared was dissolved in 25 ml of tert-amyl alcohol, followed by addition of 7.7 g (57 mmol) of K₂CO₃ and 33 ml (134 mmol) of 1-bromododecane, and stirring at 95° C. for 96 h. After removing the solids by filtration under reduced pressure, 5.5 ml of iodomethane was added, followed by stirring at 60° C. for 24 h in a sealed flask-condenser system. The resultant solution was added to excess of ethyl acetate; the precipitate formed was recovered by filtration under reduced pressure, washed with excess of ethyl acetate, and dried at r.t. under vacuum overnight. Yield: 7.0 g. ¹H NMR for 2a (CDCl₃): δ=5.5-3.0 (NCH₂CH₂(CH₂)₉CH₃, NCH₂CH₂N, NCH₃), 1.80 (NCH₂CH₂(CH₂)₉CH₃), 1.6-1.0 (NCH₂CH₂(CH₂)₉CH₃), 0.88 (NCH₂CH₂(CH₂)₉CH₃).

Polycations 2b and 2c (FIG. 1B) from linear 21.7-kDa and 2.17-kDa PEIs, respectively, were synthesized as described in the preceding paragraph, except that after the N-methylation the reaction mixture was poured into methanol to obtain the final product. ¹H NMR (CDCl₃) for 2b: δ=5.5-3.0 (NCH₂CH₂(CH₂)₉CH₃, NCH₂CH₂N, NCH₃), 1.80 (NCH₂CH₂(CH₂)₉CH₃), 1.6-1.0 (NCH₂CH₂(CH₂)₉CH₃), 0.88 (NCH₂CH₂(CH₂)₉CH₃); for 2c: δ=5.5-3.0 (NCH₂CH₂(CH₂)₉CH₃, NCH₂CH₂N, NCH₃), 1.83 (NCH₂CH₂(CH₂)₉CH₃), 1.6-1.0 (NCH₂CH₂(CH₂)₉CH₃), 0.88 (NCH₂CH₂(CH₂)₉CH₃).

N,N-Docosyl,methyl-PET (3) (FIG. 1B) was synthesized from linear 217-kDa PEI similarly to 2, except that 1-bromodocosane was used as the alkylating agent instead of 1-bromododecane. ¹H NMR (CDCl₃): δ=5.5-3.0 (NCH₂CH₂(CH₂)₁₉CH₃, NCH₂CH₂N, NCH₃), 1.85 (NCH₂CH₂(CH₂)₁₉CH₃), 1.6-1.0 (NCH₂CH₂(CH₂)₁₉CH₃), 0.88 (NCH₂CH₂(CH₂)₁₉CH₃).

N-(15-Carboxypentadecyl)-PEI (4) (FIG. 1B) HCl salt was synthesized by dissolving 86 mg (2 mmol on the monomer basis) of linear 217-kDa PEI and 670 mg (2 mmol) of 16-bromohexadecanoic acid in 10 ml of tert-amyl alcohol, followed by addition of 0.61 g (4.4 mmol) of K₂CO₃ and stirring the reaction mixture at 95° C. for 96 h. After cooling to r.t., the reaction mixture was poured into 100 ml of acetone and filtered. The filter cake was suspended in 30 ml of CH₂Cl₂ and stirred with 30 ml of 1 N HCl for 2 h. The organic phase (containing undissolved solids) was separated and filtered, and the solid residue obtained was washed with CH₂Cl₂ and dried under vacuum. The product was then dissolved in 50 ml of CHCl₃ and stirred with 40 ml of 1 N HCl for 3 h, followed by separation of the organic phase and solvent evaporation. The salt of 4 (FIG. 1B) was obtained as a pale yellow solid; yield: 0.39 g. ¹H NMR (DMSO-d₆): δ=4.0-2.8 (NCH₂CH₂N, NCH₂(CH₂)₁₄CO₂H), 2.17 (CH₂CO₂H), 1.8-1.4 (CH₂CH₂CO₂H, NCH₂CH₂(CH₂)₁₃CO₂H), 1.4-1.1 (NCH₂CH₂(CH₂)₁₁CH₂CH₂CO₂H).

N-(11-Carboxyundecanoyl)-PEI (5) (FIG. 1B). Dodecanedioic acid (4.6 g, 20 mmol) was suspended in 100 ml of dry CH₂Cl₂, followed by addition of 2.16 g (20 mmol) of benzyl alcohol, catalytic amounts of 4-(dimethylamino)pyridine, and 4.12 g (20 mmol) of 1,3-dicyclohexylcarbodiimide. After stirring the mixture for 48 h at room temperature (“r.t.”), the solid was removed by filtration, and the filtrate was washed with 60 ml of 1 N HCl. The organic phase was dried with anhydrous Na₂SO₄, and the solvent was evaporated under reduced pressure. Silica gel column chromatography (2:3 (v/v) ethyl acetate/hexane as a mobile phase) resulted in 1.5 g (24% yield) of dodecanedioic acid mono-benzyl ester. ¹H NMR spectrum (CDCl₃) was consistent with the literature data (Thomas et al. (2005) Proc Natl Acad Sci USA 102:5679-5684). Then 1.28 g (5.2 mmol) of this product was dissolved in 10 ml of dry CH₂Cl₂, followed by the addition of 0.66 g (5.2 mmol) of oxalyl chloride and one drop of N,N-dimethylformamide. After stirring the reaction mixture at r.t. for 1 h, the solvent and excess of oxalyl chloride were removed under vacuum to give the corresponding carbonyl chloride used in the next step without further purification.

Linear 217-kDa PET (86 mg, 2 mmol on the monomer basis) and N,N-diisopropylethylamine (DIPEA) (0.52 g, 4 mmol) were dissolved in 10 ml of CH₂Cl₂, and the reaction mixture was chilled to 0° C. using an ice-water bath. To this solution, the carbonyl chloride made above in 10 ml of dry CH₂Cl₂ was added dropwise, the ice-water bath was removed, and the reaction mixture was stirred at r.t. for 24 h. The reaction was quenched with 2 ml of methanol, and the solvent was evaporated. The residue obtained was washed with five 30-ml portions of methanol to remove soluble components and dried under vacuum to yield N-[(11-benzyloxycarbonyl)undecanoy]-PEI as a white solid (0.6 g, 87%). ¹H NMR (CDCl₃): δ=7.34 (m, 5H, C₆H₅), 5.10 (s, 2H, C₆H₅CH₂), 3.43 (s, 4H, NCH₂CH₂N), 2.33 (m, 4H, CH₂CO), 1.60 (m, 4H, CH₂CH₂CO), 1.26 (s, 12H, OCCH₂CH₂(CH₂)₆CH₂CH₂CO). Finally, 60 mg (0.174 mmol on the monomer basis) of this compound was dissolved in 1 ml of THF and deprotected by adding 0.5 ml of 1 N NaOH and stirring for 24 h at r.t. The solution was neutralized with 0.2 ml of 2 N HCl and the solvent was removed to give a solid residue, which was washed first with water to remove NaCl and then with CHCl₃ to remove benzyl alcohol. Yield: 40 mg (90%). ¹H NMR for 5 (CD₃OD): δ=3.43 (s, 4H, NCH₂CH₂N), 2.40-2.10 (m, 4H, CH₂CO), 1.55 (m, 4H, CH₂CH₂CO), 1.26 (s, 12H, OCCH₂CH₂(CH₂)₆CH₂CH₂CO).

N-(Undecanoyl)-PEI (6) (FIG. 1B) was synthesized by dissolving 1.08 g (25 mmol on the monomer basis) of 217-kDa linear PEI in 100 ml of chloroform, to which 6.46 g (50 mmol) of DIPEA was added. The reaction mixture was cooled to 0° C. using an ice-water bath, and 11.2 g (50 mmol) of lauroyl chloride was added dropwise over 30 min. The ice-water bath was then removed, and the reaction mixture was stirred at r.t. for 24 h. Half of the solvent was removed under reduced pressure, and the remaining solution was poured into 350 ml of methanol. After standing overnight, the solid was separated by filtration and washed with five 50-ml portions of methanol. Yield: 4.87 g (86%). ¹H NMR of 6 (CDCl₃): δ=3.43 (s, 4H, NCH₂CH₂N), 2.28 (d, 2H, COCH₂), 1.59 (s, 2H, COCH₂CH₂), 1.4-1.2 (br s, 16H, (CH₂)₈CH₃), 0.88 (t, 3H, CH₃).

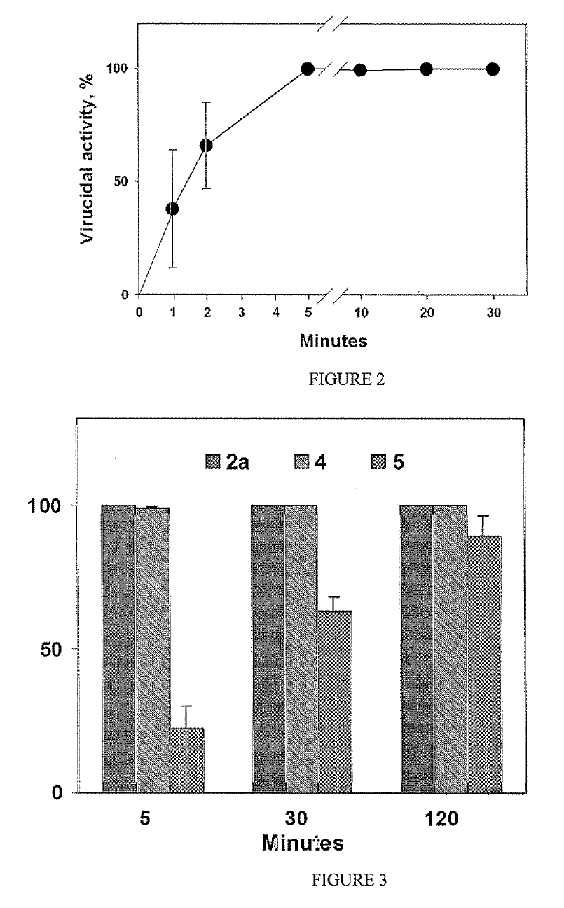

Preparation of Painted Slides. Coating polymers were dissolved (50 mg/ml) in butanol for 1a-c (FIG. 1A) and 2a-c (FIG. 1B), chloroform for 3, hot ethanol for 4, methanol-dichloromethane (1:1) for 5 (FIG. 1B), and dichloromethane for 6 (FIG. 1B) with vortexing. Commercial glass (VWR Microscope) slides, 2.5 cm×7.5 cm for bactericidal tests and 2.5 cm×2.5 cm for virucidal tests, were brush-coated with one of these solutions using a cotton swab, followed by air drying.

Determination of Bactericidal Efficiency. A 100-μl suspension of S. aureus or E. coli in 0.1 M PBS (approximately 10¹¹ cells/mL) was added to 20 ml of the yeast-dextrose broth in a 50-ml sterile centrifuge tube, followed by shaking at 200 rpm and 37° C. overnight (Innova 4200 Incubator Shaker, New Brunswick Scientific). The bacterial cells were harvested by centrifugation at 6,000 rpm for 10 min (Sorvall RC-5B, DuPont Instruments), washed twice with PBS, and diluted to 5×10⁶ cells/ml for S. aureus and to 3×10⁷ cells/ml for E. coli. The bacterial suspensions in PBS were sprayed onto slides at a rate of approximately 10 ml/min in a fume hood. After a 2-min r.t. drying under air, the resultant slide was placed in a Petri dish and immediately covered with a layer of solid growth agar (1.5% agar in the yeast-dextrose broth, autoclaved, poured into a Petri dish, and allowed to gel at r.t. overnight). The Petri dish was sealed and incubated at 37° C. overnight, and the bacterial colonies grown on the slide surface were counted on a light box.

Preparation of Viruses in Chicken Eggs. A 100-μl aliquot of a 10-fold diluted solution of viruses (CDC samples) was injected into the allantoic fluid of 10-day-old embryonated chicken eggs. The eggs were subsequently incubated at 37° C. for 48 h and then at 4° C. for 24 h. The allantoic liquid was collected and centrifuged at 1,200 rpm at 4° C. for 20 min, followed by passing the supernatant through a 0.45-μm syringe filter (low protein binding). The supernatant was stored at −80° C. The virus titer was determined by the plaque assay as described below.

Plaque Assay. Confluent MDCK cells in 6-well cell culture plates were washed twice with 5 ml of PBS and infected with 200 μl of a virus solution in phosphate buffered saline (PBS) at room temperature. for 1 h. The solution was then removed by aspiration, and the cells were overlaid with 2 ml of plaque medium (6.9 ml of 2×F12 medium supplemented with 139 μL of 0.01% DEAE-dextran, 277 μL of 5% NaHCO₃, 139 μL (100 U/ml) penicillin G, 100 μg/ml streptomycin, 122 μL of trypsin-EDTA, and 4.2 mL of 2.0% agar (Oxoid Co., purified agar, L28). After a 3-day incubation at 37° C. in a humidified-air atmosphere (5% CO₂/95% air), the cells were fixed with 1% aqueous formaldehyde for 1 h at room temperature. The agar overlay was removed, and the cells were stained with 0.1% Crystal Violet in 20% (v/v) aqueous methanol for 2 min at room temperature. After removing the excess of the dye by aspiration, the plaques were counted.

Virucidal Activity. A glass slide coated with polymer (or uncoated in a control experiment) was placed into a polystyrene Petri dish (6.0 cm×1.5 cm), and then a 10-μl droplet of a 10⁵-10⁷ pfu/ml virus solution in phosphate buffered saline (PBS) was deposited in the center of the slide. A second, uncoated glass slide was put on top and pressed to spread the droplet between the slides. This “sandwich” system was incubated at room temperature typically for 5 minutes. One edge of the top slide was then lifted, and virus-exposed sides of both slides were thoroughly washed with 0.99 ml of PBS. Finally, plaque assay was performed to determine the virucidal activity of the washings and of their 2-fold serial dilutions (5 times) for the coated slide. A 100- to 200-fold additional dilution of the washing solution, followed by 2-fold serial dilutions (5 times) was made to perform the plaque assay for the uncoated slide (control).

Non-leaching Tests. No. 1: A glass slide coated with a polymer (or plain in a control experiment) was placed upside down in a well of a 6-well plate containing 2 ml of PBS and incubated for 2 h at r.t. with periodic agitation. Then 0.99 ml of the solution was withdrawn, mixed with 10 μl of a virus solution [(1.4±0.1)×10⁷ pfu/ml of WSN] and incubated at r.t. for 30 min. After a 200-fold dilution and subsequent 2-fold serial dilutions (5 times), the plaque assay was performed as described above.

No. 2: 200 mg of a neat solid polymer was dispersed in 1 ml of PBS by vortexing for 5 min and then it was incubated at r.t. for 16 h, followed by centrifugation at 9,000 rpm (VWR Scientific Products, Galaxy 7) for 30 min thrice and then passing through a glass wool to obtain a clear solution. Then 0.39 ml of this solution was mixed with 10 μl of a virus solution [(8.7±1.4)×10⁶ pfu/ml of WSN] and incubated at r.t. for 30 min. After a 300-fold dilution and subsequent 2-fold serial dilutions (5 times), the plaque assay was performed as described above.

Results

To mimic a scenario whereby aerosolized aqueous droplets containing influenza virus settle onto surfaces and the virus then spreads (Wright et al. (2001) in Fields Virology, 4^{th} edition, eds. Knipe D M, Howley P M (Lippincott, Philadelphia, Pa.), pp 1533-1579), the following approach was utilized. A 10-μl droplet of a PBS-buffered solution containing 1.6±0.3)×10³ plaque-forming units (pfu) of the A/WSN/33 (H1N1) strain of influenza virus was placed in the center of a 2.5 cm×2.5 cm glass slide (either coated or plain control). Then another, plain glass slide of the same size was placed on top and pressed against the first to flatten the droplet. After a r.t. incubation for 30 min (unless stated otherwise), one edge of the upper slide was lifted and both virus-exposed glass surfaces were thoroughly washed with 1.99 ml of aqueous PBS. The resultant washings underwent five consecutive 2-fold dilutions with the same buffer, and 200-μl aliquots of the undiluted and the serially diluted samples were each added into a well of a E-well plate covered with a monolayer of Madin-Darby canine kidney (MDCK) cells. After an 1-hr incubation, the solutions were removed, and 2 ml of plaque medium was placed in each well, followed by a 3-day incubation at 37° C. in a humidified air. Finally, the cells were fixed with formaldehyde, stained following removal of the agar overlay, and the plaques were counted.

When this procedure was applied to uncoated slides, the concentration of the viable virus in the washings barely changed compared to the identically diluted droplets not exposed to the slide: 650±150 vs. 800±150 pfu/ml, respectively. Thus, such a contact with a control glass slides results in no statistically significant decrease in the viral titer, i.e., influenza virus survives essentially unscathed in this incubation at r.t. between two plain glass slides.

Next, a glass slide was painted with a solution of branched N,N-dodecyl,methyl-PEI (1a) (synthesized by quaternizing a branched 750-kDa PEI as depicted in FIG. 1) in butanol and the solvent allowed to evaporate. When the foregoing testing was employed with this coated slide, not a single plaque was detected even using the undiluted washings. To further quantify this apparent 100% virucidal activity, a separate experiment was carried out with a higher initial viral titer and also a lower dilution. Despite the greater sensitivity and assay range, still no plaques were observed, indicating that the exposure of the virus to the coated slides for 30 min lowers its titer at least some 10,000 fold (i.e., 4 logs).

When PEI precursors of lower than 750 kDa molecular weights, namely 25 kDa and 2 kDa (FIG. 1A), were employed to make the hydrophobic polycationic coatings (1b and 1c, respectively, FIG. 1A), very high but slightly incomplete virucidal efficiencies were observed—98±0.4% and 97±0.2%, respectively. It is noteworthy that slides painted with these smaller N-alkylated PEI derivatives were previously found to also have incomplete bactericidal efficiencies (Park et al. (2006) Biotechnol. Progr., 22:584-589). Thus, as in the case of bacteria, the polycations must be large enough, perhaps to allow their tentacles to penetrate and damage the viral lipid envelope.

For simple steric reasons, the chain length constraints should be alleviated by replacing the branched polycations with their linear counterparts. To test this hypothesis, the virucidal properties of three linear N,N-dodecyl,methyl-PEIs—2a, 2b, and 2c (FIG. 1B), synthesized from the 217-kDa, 21.7-kDa, and 2.17-kDa linear PEI precursors, respectively, were tested. Slides coated with all of these linear hydrophobic polycations indeed inactivated influenza virus with a 100% efficiency. Moreover, 2a (like 1a) was shown to reduce the viral titer by at least some four logs; it was used in most subsequent experiments.

To further investigate the effect of hydrophobicity of the polycation in virucidal action, we raised the latter by alkylating linear 217-kDa PEI with docosyl (C₂₂) instead of dodecyl (C₁₂) bromide (FIG. 1). A glass slide coated with resultant 3 (FIG. 1B) was as completely lethal to influenza virus as that coated with 1a (FIG. 1A) or 2a-c (FIG. 1B).

To determine how quick the virucidal action is in our experimental system, the time of exposure of influenza virus to a slide coated with 2a (FIG. 1B) was varied from 1 min to 2 hr. As seen in FIG. 2, a 100% virucidal efficiency is already achieved after as little as 5 min, albeit not 1 or 2 min, possibly reflecting the time required for all viral particles present to reach the coated surface.

All the coating paints examined thus far were polycationic. To ascertain the role of the charge, derivatives of linear 217-kDa PEI were synthesized that were nominally zwitter-ionic (4), anionic (5), and electrostatically neutral (6) with otherwise roughly similar side chains as in 1 and 2 (FIG. 1). As shown in Table 1 (second column), zwitter-ionic 4, as cationic 1a and 2a (and also 2b-c and 3, see above), is 100% virucidal after a 30-min exposure. In contrast, the anionic 5 (FIG. 1B) is only partially virucidal, and the neutral 6 (FIG. 1B) not at all. The virucidal impotence of the last one is presumably owing to the lack of individual sticking-out tentacles which, in the absence of significant charges, should strongly hydrophobically associate with each other. That the polyanionic coating significantly inactivates influenza virus suggests that there are both positively and negatively charged sites attacked in the viral membrane; the latter ones appear predominant because 2a-c (FIG. 1B) and even 4 (FIG. 1B) are virucidally superior to 5 (FIG. 1B).

TABLE 1
Microbicidal activity of glass slides painted with 1a, 2a, 4, 5, and 6.

| | | |
|---|---|---|
| PEI | Virucidal activity^{a} | Bactericidal activity, |

| derivative % | after 30 min, % | S. aureus | E. coli |
|---|---|---|---|
| 1a | 100 | 99 ± 1^{b} | 99 ± 1^{b} |
| 2a | 100 | 100 | 100 |
| 4 | 100 | 26 ± 4 | 14 ± 2 |
| 5 | 66 ± 3 | 21 ± 1 | 22 ± 3 |
| 6 | 6 ± 6 | 34 ± 1 | 14 ± 2 |

^{a}Virucidal activities were tested against the WSN strain of influenza virus.
^{b}Glass slides used in these experiments are painted twice or more to attain the levels of activity indicated (presumably reflecting imperfections of our painting procedure).

Painting a glass slide with branched or linear N,N-dodecyl,methyl-PEIs and certain other hydrophobic PEI derivatives enables it to kill influenza virus with essentially a 100% efficiency (at least a 4-log reduction in the viral titer) within minutes, as well as the airborne human pathogenic bacteria Escherichia coli and Staphylococcus aureus. For most of the coating polyions this virucidal action is shown to be on contact, i.e., solely by the polymeric chains anchored to the slide surface; for others, a contribution of the polyion leaching from the painted surface cannot be ruled out. A relationship between the structure of the derivatized PEI and the resultant virucidal activity of the painted surface has been elucidated.

To gain further insights into these observations, the time course of the virucidal activity of slides coated with 4 and 5 (FIG. 1B) was examined. Not only did zwitter-ionic 4, like cationic 2a (FIG. 1B), already inactivate the entirety of the exposed influenza virus after a 30-min incubation, but even after just 5 min the 4's virucidal activity was as high as 98±0.7% (FIG. 3). The virucidal activity of anionic 5 rose steadily with time (the last bar at each time point in FIG. 3) to reach 89±7% after a 2-h exposure. Thus it seems that the differences in virucidal activities among the polymeric coatings are a matter of kinetics rather than ultimate degree, i.e., that the hydrophobic polycations merely inactivate the virus faster than other hydrophobic polyions.

The leaching conditions into a 10-0 aqueous droplet squeezed between a coated and plain glass slides were estimated as follows: A coated slide was placed upside down in a well of a 6-well plate containing 2 ml of a PBS-buffered solution and incubated for 2 h (the longest exposure employed in this study, e.g., see FIG. 3) with periodic agitation to facilitate mass transfer. Then to 0.99 ml of this solution 10 μl of an influenza virus solution was added, followed by a 30-min incubation at r.t., appropriate dilutions, and the standard viral assay. With glass slides coated with 1a, 1b, 2b, 3, 4, 5, and 6 (FIG. 1) the viral titers measured were statistically indistinguishable from that determined when the uncoated slide was subjected to the same procedure. In contrast, when the polycations 1c, 2a, and 2c (FIG. 1) were used as coatings, the viral titers obtained were 20% to 40% below that with the uncoated slide.

In the second set of controls, the possible extent of leaching of the polymers deposited onto the glass slide surface was increased. To this end, 200 mg of a neat solid polymer was dispersed in 1 ml of an aqueous PBS by vortexing, followed by a 16-h incubation at r.t. and subsequent centrifugation to obtain a clear solution. To 390 μl of this solution, 10 μl of an influenza virus solution was added, incubated for 30 min at r.t., appropriately diluted, and titrated for the virus. Even in this exaggerated leaching test, with 1b, 2b, 3, 5, and 6 (FIG. 1) as coatings the viral titer obtained was statistically indistinguishable from that observed when 390 μl of a fresh aqueous PBS was employed instead of those putatively saturated with the polymers (with 1a, 1c, 2a, 2c, and 4, the viral titers were much lower).

On the basis of the results of the foregoing controls it was concluded that at least for slides painted with 1a, 1b, 2b, 3, 4, and 5 (FIG. 1) the virucidal activity observed is solely due to the polyions remaining deposited on the slide's surface, i.e., the tentacles of these immobilized polyions inactivate the virus on contact. In contrast, in the case of 1c, 2a, and 2c (FIG. 1) coatings, contributions of the leached polycations to the virucidal activity of the painted slides cannot be ruled out.

The bactericidal activities of the differently charged derivatives of linear 217-kDa PEI against two common human pathogenic bacteria—Gram-positive Staphylococcus aureus and Gram-negative Escherichia coli were also compared. Slides painted with 1a and 2a (FIG. 1A) killed both airborne bacteria on contact with a 100% efficiency or statistically indistinguishably from that level (Table 1, the last two columns). In contrast, 4, 5, and 6 (FIG. 1B) coatings were only marginally bactericidal (even though the first one is completely virucidal).

To ascertain the generality of the ability of 1 and 2 (FIG. 1) to inactivate influenza virus, the coatings were tested against A/Victoria/3/75 (H3N2), a strain distinct from the A/WSN/33 (H1N1). Slides painted with 1a and 2a (FIG. 1) both exhibited 98±0.5% virucidal activities after a 30-min exposure to the coated surfaces and 100% virucidal activities after 2 h. Therefore, although the Victoria strain appears more resistant than its WSN counterpart, given enough time, 1a and 2a (FIG. 1) coatings completely inactivate both of them.

The results demonstrate that certain hydrophobic polycations can be painted onto surfaces to render them not only highly bactericidal but also extremely virucidal against at least two distinct strains of influenza virus and presumably other enveloped viruses. In terms of its virucidal and bactericidal efficiencies, as well as the lack of ambiguity in the virucidal mode of action, painting with 1a seems optimal. Given the simplicity of the coating procedure, it should be applicable to various common materials, thereby enabling them to interrupt the spread of both viral and bacterial infections.

The antiviral activity of N,N-docecyl,methyl-PEI against human A/Wuhan/359/95 (H3N2)-like influenza virus, avian A/turkey/Minnessota/833/80 (H4N2) influenza virus, and their drug resistant variants was also evaluated. After a 5 minute exposure of an aqueous solution of A/turkey/Minnessota/833/80 (H4N2) to an uncoated glass surface, numerous plaques were clearly visible when MDCK cells were infected with 200 μl of the 200-fold diluted washing solution. In contrast, when 200 μl of the undiluted washing solution (after exposure to a glass slide painted with N,N-docecyl,methyl-PEI) was used to infect MDCK cells, no plaques were observed. Quantification of this data is shown in Table 2.

Table 2 depicts the results of a 5-min exposure of the virus solutions either to an uncoated glass slide (a control) or to that painted with N,N-dodecyl,methyl-PEI. While the exposure to the control slide only marginally affects the viral titer after accounting for dilution, the polycation-painted slides completely inactivated the exposed influenza virus reducing its titer over 3,000 times.

TABLE 2
Virucidal activity of glass slides painted with N,N-dodecyl,methyl-
polyethylenimine against wild-type strains of a human influenza
A Wuhan (H3N2) and an avian influenza A turkey (H4N2) virus

| | | Final Viral Titer | | |
|---|---|---|---|---|
| | | (pfu/ml)^{a} | | |
| | Initial Viral | | Coated | Virus Titer |
| Strain | Titer (pfu/ml) | Uncoated Slide | Slide | Reduction |
| A/Wuhan/ | (4.8 ± 0.5) × 10⁵ | (3.1 ± 0.4) × 10³ | 0 | 100% (>3.5 |
| 359/95 | | | | logs) |
| A/turkey/ | (6.1 ± 1.1) × 10⁶ | (3.7 ± 0.4) × 10⁴ | 0 | 100% (>4.5 |
| MN/833/ | | | | logs) |
| 80 | | | | |

^{a}After a 5 minute exposure and washing (100-fold dilution) with phosphate-buffered saline (PBS)

Although two neuraminidase inhibitors, oseltamivir and zanamivir, were introduced commercially several years ago to treat influenza A infections a growing concern with their use is the development of drug-resistant virus strains and their subsequent transmission. In fact, several neuraminidase mutants, Glu119Gly, Glu119Ala, Glu119Asp, and Arg292Lys, with diminished drug susceptibility have been isolated by using zanamivir in vitro. Furthermore, a mutant (Arg152Lys) influenza strain with a lowered drug sensitivity has been recovered from an immuno-compromised person treated with zanamivir. Likewise, mutations in the neuraminidase glycoprotein (Glu119Val, His274Tyr, and Arg292Lys) causing resistance to oseltamivir have arisen both in challenge studies and in patients with naturally acquired infections.

Therefore, it was important to ascertain whether N,N-dodecyl,methyl-PEI-coated surfaces can kill drug-resistant strains of influenza A virus in addition to their wild-type parental strains. The antiviral activity of coated slides to a zanamivir-resistant strain of avian influenza virus A/turkey/MN/833/80, Glu119Asp, was investigated. After a 5-min exposure of an aqueous solution of this viral strain to an uncoated glass surface, numerous plaques were clearly visible when MDCK cells were infected with 200 μL of the 200-fold diluted washing solution. In contrast, when 200 μL of even the undiluted washing solution after the analogous exposure to a glass slide painted with the hydrophobic polycation was used to infect MDCK cells, no plaque formation was observed. Quantification of these data (see Table 3) reveals at least a 100,000-fold decrease in the viral titer due to the exposure to the polycation-coated surface, as compared to the uncoated one.

TABLE 3
Virucidal activity of glass slides painted with N,N-dodecyl,methyl-polyethylenimine
against drug resistant strains of human influenza A Wuhan (H3N2) and avian
influenza A turkey (H4N2) virus

| | | | Final Viral Titer (pfu/ml) | | |
|---|---|---|---|---|---|
| | | Initial Viral Titer | | Coated | Virus Titer |
| Strain | Resistance Against | (pfu/ml) | Uncoated Slide | Slide | Reduction |
| A/turkey/MN/833/80 | zanamivir | (2.7 ± 0.6) × 10⁷ | (1.5 ± 0.2) × 10⁵ | 0 | 100% (>5.1 |
| (Glu119Asp)^{a} | | | | | logs) |
| A/turkey/MN/833/80 | zanamivir | (1.7 ± 0.6) × 10⁶ | (1.0 ± 0.2) × 10⁴ | 0 | 100% (>4.0 |
| (Glu119Gly)^{a} | | | | | logs) |
| A/Wuhan/359/95 | oseltamivir | (1.2 ± 0.6) × 10⁶ | (7.8 ± 0.6) × 10³ | 0 | 100% (>3.9 |
| (Glu119Val)^{a} | | | | | logs) |
| A/turkey/MN/833/80 | zanamivir and | (2.9 ± 0.3) × 10⁶ | (1.8 ± 0.4) × 10⁴ | 0 | 100% (>4.2 |
| (Arg292Lys)^{a} | oseltamivir | | | | logs) |

^{a}All mutations are in the neuraminidase glycoprotein.

Similar results were obtained with a different neuraminidase mutant of the zanamivir-resistant avian virus, Glu119Gly, as well as with the Glu119Val neuraminidase mutant of the oseltamivir-resistant human virus (second and third entries in Table 3). Finally, even with a strain of the avian influenza virus which is resistant to both zanamivir and oseltamivir (the Arg292Lys neuraminidase mutation), a brief exposure to a surface painted with N,N-dodecyl,methyl-PEI results in over a 10,000-fold drop in the viral titer (the last entry in Table 3).

All publications and patents mentioned herein are hereby incorporated by reference in their entirety. Those skilled in the art will recognize, or be able to ascertain using no more than routine experimentation, equivalents to the specific embodiments described herein. Such equivalents are intended to be encompassed by the following claims.

We claim:

1. A virucidal composition comprising a coating comprising a hydrophobic, water-insoluble polymer deposited on an inert surface, wherein if the polymer is a cationic polymer, the polymer is selected from the group consisting of polymers of Formula I 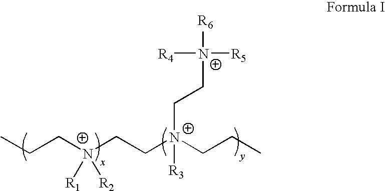
wherein R₁-R₆ are alkyl groups,
polymers of Formula II 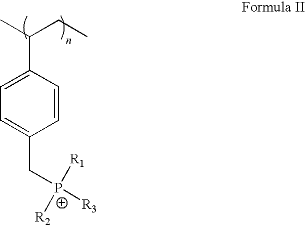
wherein R₁-R₃ are alkyl groups, and
polymers of Formula III, 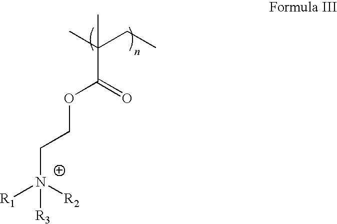
wherein R₁-R₃ are alkyl groups.

2. The composition of claim 1, wherein the coating associates with the surface via non-covalent interactions.

3. The composition of claim 1, wherein the polymer is anionic.

4. The composition of claim 1 wherein the polymer is zwitterionic.

5. The composition of claim 1 wherein the polymer has a molecular weight of at least 20 kDa.

6. The composition of claim 1, wherein the polymer has a molecular weight of at least 50 kDa.

7. The composition of claim 1, wherein the polymer has a molecular weight of at least 100 kDa.

8. The composition of claim 5, wherein the polymer is a zwitterionic polymer having a structure selected from the group consisting of:

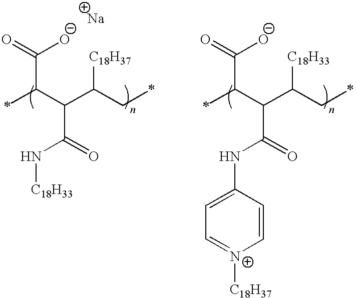

9. The composition of claim 1, wherein the coating is applied to the surface by painting, brushing, dipping, or spraying.

10. The composition of claim 1, wherein the surface is formed of a material selected from the group consisting of metals, ceramics, glass, polymers, plastics, and fibers.

11. The composition of claim 1, wherein the surface is the surface of a device or implant to be placed into a body or tissue.

12. The composition of claim 1, wherein the surface is the surface of a fabric, gauze, tissue, surgical drape, air filter, tubing, or surgical instrument.

13. The composition of claim 1, wherein the surface is the surface of a toy, a bathroom fixture, countertop, tabletop, handle, computer, military gear, clothing, paper product, window, door, or interior wall.

14. A method for killing viruses comprising providing the composition of claim 1.

15. The method of claim 14, wherein the virus is an enveloped virus.

16. The method of claim 14, wherein the virus is influenza.

17. The method of claim 14, wherein the surface is the surface of a material selected from the group consisting of metals, ceramics, glass, polymers, and fibers.

18. The method of claim 14, wherein the surface is the surface of a device or implant to be placed into a body or tissue.

19. The method of claim 14, wherein the surface is the surface of fabric, gauze, tissue, surgical drape, air filter, tubing, or surgical instrument.

20. The method of claim 14, wherein the surface is the surface of a toy, a bathroom fixture, countertop, tabletop, handle, computer, military gear, clothing, paper product, window, door, or interior wall.
